(19) United States
(12) Patent Application Publication
(10) Pub. No.: US 2009/0036753 A1
(43) Pub. Date: Feb. 5, 2009

(54) CONTINUOUS GLUCOSE MONITORING-DIRECTED ADJUSTMENTS IN BASAL INSULIN RATE AND INSULIN BOLUS DOSING FORMULAS

(76) Inventor: Allen B. King, Salinas, CA (US)

Correspondence Address: SHAY GLENN LLP, 2755 CAMPUS DRIVE, SUITE 210, SAN MATEO, CA, 94403, US

(21) Appl. No.: 11/831,623

(22) Filed: Jul. 31, 2007

(51) Int. Cl. A61B 5/00

(52) U.S. Cl. 600/301; 128/898

(57) ABSTRACT

A method for individualized management of diabetes in insulin-dependent patients provides a period of evaluation as the patient adheres to a structured pattern of eating, sleeping, and physical activity. Glucose is monitored with a continuous glucose monitoring system, insulin doses are metered, carbohydrate consumption is quantified, and glucose, carbohydrate, and insulin data are collected and analyzed. Insulin dosage is adjusted in three steps: (1) an insulin dosage is estimated from conventional formulas, (2) adjustments are made according to the patient's clinical specifics, and (3) further insulin dose adjustments are made according to glucose data obtained during the evaluation period. By the end of the evaluation period, substantially normal glucose values are achieved, and quantitative relationships from data are calculated that are then applied to determine insulin dosages for an ensuing period of therapy. By this method, diabetic patients achieve a near normal glycemic profile, and without significant occurrence of hypoglycemic episodes.

### FIELD OF THE INVENTION

The invention is in the field of medical methods related to managing the treatment of insulin-treated diabetic subjects, more particularly to adjusting an insulin dosage schedule so as to achieve glycemic control.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

Since the earliest use of insulin for treatment of diabetes, efforts have been made to adjust the dosage based on clinical experience, and more particularly on measurements of the level of glucose. Initially the glucose tests were done infrequently and in a standard clinical laboratory. With the advent in the 1980's of intermittent self-monitored glucose testing, such testing was able to be done by the patient and with a frequency of up to seven times per day without great cost. The application of information derived from more frequent glucose testing has indeed allowed significantly better glucose control, and has lowered the occurrence of complications due to poor glycemic control. In 1999, MiniMed (now Medtronic Minimed) developed a continuous glucose monitor that could deliver glucose readings every five minutes for a period of up to three days. The results were not provided to the patient directly by the monitor, but rather were provided in the setting of doctor's office. Beginning in the 2000's Medtronic and other companies have provided wireless monitors that allow glucose determinations every 1-5 minutes that display to the patient, and variously provide indications of the trend of the glucose as well as high-glucose and low-glucose alarms. Technological advances have been made also in the development of insulin pumps, which can replace multiple daily self-injections of insulin. These now-available devices can deliver precise insulin dosages, typically on a programmable schedule which may be adjustable on the basis of input from the user or healthcare professional, or on the basis of data from a continuous glucose monitor.

Basic algorithms have been developed that estimate an appropriate insulin dosing schedule based, for example, on patient weight, and these algorithms provide a reasonable first approximation of a clinically appropriate insulin dosing schedule. There is, however, considerable variation among patients with regard to their metabolism and responsiveness to insulin, and thus a generic first approximation of an insulin dosing schedule is but an approximation for the hypothetical average patient, and may not be clinically satisfactory for the individual patient.

Various approaches have been applied to making calculations that use continuous glucose monitor (CGM) data to improve or adjust insulin dosing. Some approaches, for example, use a glucose level trend analysis that provides a quick response to changes in glucose as a result of patient eating or activity behavior (with provider or an electronic machine augmentation) by way of making appropriate adjustments in insulin dosage. Other approaches, for example, provide for setting a basal insulin dose or meal-related insulin bolus requirement based on consideration of a patient's history, particularly glucose excursion data over a period of time.

Nevertheless, in spite of current aspects of diabetes care management such as (1) a high level of understanding of the dynamics of insulin and glucose, and the role of factors such body weight and other clinical variables that can affect the clinical appropriateness of insulin dosage schedules, (2) the availability of high quality glucose data from continuous glucose monitors, and (3) the availability of highly controllable insulin dosing from insulin pumps, an ability to dependably establish euglycemia with clinically appropriate insulin dosing in the individual diabetic patient has yet to be satisfactorily achieved.

### SUMMARY OF INVENTION

The invention relates to a therapeutic method for the management of diabetes care in insulin-dependent subjects that monitors glucose levels continuously and makes insulin-dose adjustments accordingly. Embodiments of the invention provide for the logging and analytical evaluation of daily continuous glucose monitoring (CGM) data, and for the generation of continuous glucose-driven-insulin adjustments (CGIA) that are delivered to the patient's insulin pump to achieve control glucose to a near normal level, without causing hypoglycemia. Typical continuous glucose monitors sample subcutaneous interstitial fluid, while typical patient self-testing draws blood from finger sticks or other sites. While minor differences may exist among glucose levels as measured in blood, plasma, or interstitial fluid, in the description herein, glucose levels from any of these sample fluids will be referred to generically, without specificity to their fluid source.

The invention relates to a method of treating a diabetic patient with an insulin dosage schedule clinically appropriate for that individual patient. Embodiments of the method include testing the patient to determine the patient's individualized carbohydrate-to-insulin ratio (CIR) and the correction factor (CF) during an evaluation period under conditions of controlled or structured daily activity, applying the CIR and CF to determine a clinically appropriate basal insulin dosage and insulin bolus dosage, and applying the insulin dosages to the patient for an ensuing period of insulin therapy.

In embodiments of the method that include testing to determine the CIR and CF include making a first estimate of an appropriate insulin dosage schedule based on available information, using that estimate at the outset of the evaluation period, and making adjustments to the first estimated dosage schedule based on continuous glucose data obtained during the evaluation period. In some of these embodiments, estimating an appropriate insulin dosage includes making a first estimate of the total basal dosage based on a formula based on any of patient weight or on the existing total daily dosage schedule for the patient. Within these embodiments, making a first estimate may include adjusting the estimated basal dosage to account for any other diabetes-related clinical factors that may affect insulin sensitivity. In some embodiments of the method, the evaluation period includes making adjustments to the first estimated dosage schedule based on continuous glucose data obtained during the evaluation period. In these latter embodiments, the data may include any one or more of observed symptoms of hyperglycemia, observed symptoms of hypoglycemia, or glucose data obtained from the continuous glucose monitor.

Embodiments of the method that include having a controlled or structured pattern of daily activity, such controlled daily activity may include any one or more of a controlled pattern of eating, a controlled pattern of physical activity, and a controlled pattern of sleep. Included as a part of controlled or structured daily activity is the keeping of a record or diary of such activity, as described and summarized and further below. Typical aspects of controlled daily activity include moderation and consistency with regard to the daily activity. For example, consistency with regard to eating may include eating a consistent number of calories from day to day. Moderation with regard to eating or food consumption may include consuming a number of calories daily that is about equal to the number of calories expended daily. Moderation with regard to eating may include eating a consistent number of calories per day, and may further include eating meals at about the same time every day. Aspects of practicing consistency with regard to physical activity may include exercising at about the same time every day, and for about the same amount every day.

Embodiments of the above described method may further include accepting or rejecting the individual as an appropriate patient for the therapy based on various screening criteria. Such criteria may include positive selection criteria related to the ability of the patient to manage the basic aspects of treating diabetes. The criteria may further include exclusionary patient selection criteria. The method may further include the patient becoming educated regarding medical components of the diabetes therapy, such education may come through engagement of health care professionals and educators, and may further include self-study on the part of the patient.

Embodiments of the invention provide for the patient being educated with regard and trained in the proper technique to operate a glucose monitor, which is a typical approach to glucose testing as provided by embodiments of the invention, as well as being trained in self-monitored glucose-testing, which is typically performed four times per day. Patients are further trained in a method of “counting carbohydrates”, so that they can accurately estimate the carbohydrate content of a meal that they eat. The patients are further educated with regard to medical aspects of insulin treatment with either a regimen of multiple daily injections (MDI) or as delivered by an insulin pump; and they are further trained in a practical sense, so that they attain a level of appropriate confidence and self-sufficiency. Patients are further provided a conceptual understanding in the theory underlying the concept of basal bolus dosing, and the medical desirability and benefits of glucose control that is as close to physiological ideal as possible.

The above-mentioned positive selection criteria for patients appropriate for the inventive method may include any one or more of patient familiarity with basic diabetes management, patient ability to count carbohydrates, the patient having knowledge about basal and bolus insulin dosing, and/or the patient having sufficient capability to self-monitor glucose with a continuous glucose monitor. The above-mentioned patient exclusionary criteria may include any one or more of demonstrated noncompliance, an impairment that comprises an ability to operate the method, an insufficient knowledge of how to use of an infusion pump (if using a pump), any clinical indications of unstable insulin sensitivity, any unstable eating or activity patterns, and/or a diabetes-complicating medical condition, particularly a digestive tract condition such as gastroparesis.

In broad aspect, the inventive method may include testing the patient during an evaluating period prior to initiating a period of insulin therapy, during which time results from the evaluating period set the insulin dosages for the ensuing therapeutic period. The method may include testing the patient to determine the patient's CIR and CF during the evaluation period, more specifically including estimating an insulin dosage by conventional approaches, individually adjusting the conventional insulin dosage to account for any individual clinical particulars of the patient, and adjusting the insulin dosage according to continuous glucose data obtained during the testing period.

Further, in broad aspect, the inventive method may include an active role for the patient, a role for an overseeing healthcare professional, and a role for mathematical rendering of collected insulin and glucose data in order to calculate a clinically appropriate insulin dosage schedule. The patient, for example, is responsible for a sufficient gaining and application of knowledge and training regarding diabetes and its management, and for compliance with guidelines, as detailed herein. Compliance may also be considered to include the application of the diabetes management-related knowledge and training. The healthcare provider, for example, is responsible for patient preparation and selection, administering the evaluation period, analyzing data, exercising appropriate clinical judgment regarding evaluating the medical aspects of the patient, particularly with regard to the glycemic condition of the patient, and combining judgment, data, and calculations into the development of an individually appropriate insulin dosage schedule.

Embodiments of the method provide for adjustment of insulin dosing that result in near normal glucose control in type 1 diabetic patients treated with an insulin pump, as described in a prospective insulin dosing study (King and Armstrong, Journal of Diabetes Science and Technology 1, 36-46, 2007). Embodiments further provide a diary of activity and eating, and hypoglycemic symptoms. Embodiments further provide a structured plan for activity and eating. Finally, embodiments of the invention provide a daily analysis of the CGM tracing, with adjustments computed by mathematic formulas in a sequence that begins with the establishment of basal insulin infusion rate, and then the adjusts the rate with an individualized carbohydrate-to-insulin ratio, and finally, with an individualized correction factor.

By the practice of this invention, the insulin dosage for insulin-treated diabetic patients may be adjusted in a patient-specific manner such that the glucose levels are stabilized and near normal in comparison to non-diabetic subjects.

### BRIEF DESCRIPTION OF THE DRAWINGS

FIG. 1 shows the relationship between total basal dose and total daily dose.

FIG. 2 shows the relationship between total basal dose and body weight.

FIG. 3 shows the relationship between the insulin correction factor (ICR) and the reciprocal of the total daily dose (1/TDD).

FIG. 4 shows the relationship between the correction factor (CF) and the reciprocal of the total daily dose (1/TDD).

FIG. 5 shows the relationship between the correction factor (CF) and the insulin to carbohydrate ratio.

FIG. 6 is a schematic depiction of an aspect of the method where insulin dosage is estimated, then subjected to a pre-dose adjustment, and to a post-dose adjustment.

FIG. 7 is a reference table of carbohydrate content of variations of breakfast, lunch, and dinner meals.

FIG. 8 is chronological graph of glucose values from a hypothetical patient whose basal glucose level is too high.

FIG. 9 is chronological graph of glucose values from a hypothetical patient whose basal glucose level is too low.

FIG. 10 is a chronological graph of glucose values from a hypothetical patient whose pre-meal glucose range is at a target value, but whose bolus glucose level is out of range.

FIG. 11 is a chronological graph of glucose values from a hypothetical patient whose glucose values both pre-meal and post-meal are too high.

### DETAILED DESCRIPTION OF THE INVENTION

The invention relates to a method for the individualized management of diabetes care in insulin-treated subjects in which a patient-specific accounting of the interrelationships among insulin doses, carbohydrate consumption, and glucose levels are determined during an insulin adjustment or evaluation period. During this evaluation period, glucose data are collected by a continuous glucose sensing system, and the results are used in making adjustments to the insulin dosage; accordingly this aspect of the method may be referred to as continuous glucose-based insulin adjustment (CGIA). The advantages provided by continuous glucose monitoring data include the accuracy of the values and the chronological density of the data that, as a whole, provide a far more accurate picture of the glucose profile of a patient than do samples being individually taken by the patient, however frequently, over the course of the day. Continuous glucose monitors further provide the analytical advantage of storing and being able to deliver data in an electronic form that lends itself well to analysis.

From this evaluation period, patient-specific mathematical constants that characterize the data are determined with regard to clinically appropriate levels of basal and bolus insulin dosages. These dosages are then applied over the course of an extended period of therapy that follows the evaluation period. By embodiments of this inventive method of insulin dosage adjustment, basal doses or rates of infusion are typically lower than conventional basal doses, and bolus doses are typically larger than conventional bolus doses. Accordingly, by embodiments of this method, the ratio of the bolus dosage to the total daily dosage (basal plus bolus) is higher than that of conventional dosages.

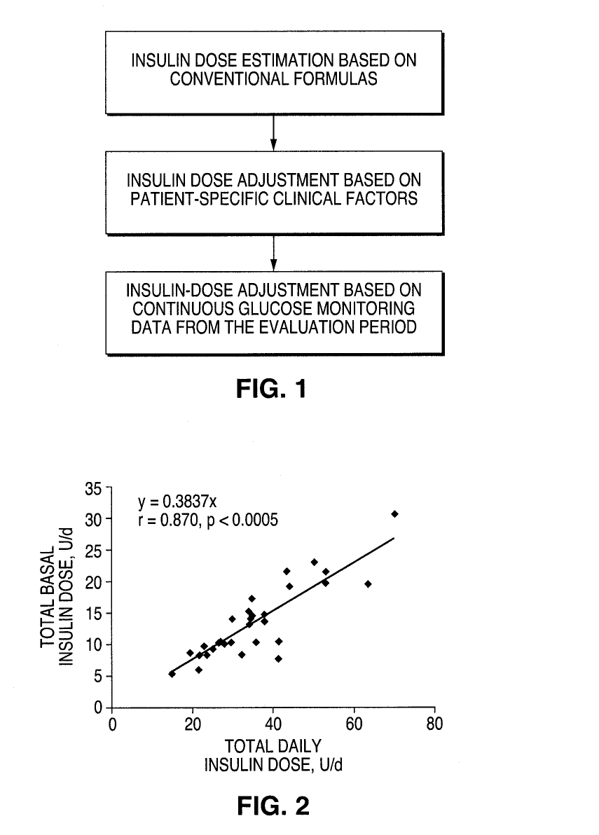

The evaluation period is of relatively short duration; a period of about four to about seven days is typically sufficient. During the evaluation period the patient adheres to a structured or controlled pattern of eating, sleeping, and other activities of daily living, glucose is closely monitored with a continuous glucose monitoring system, insulin doses are closely measured, and insulin dosage and glucose data are collected and analyzed. In brief, as shown in FIG. 1, (1) the insulin dosage for a patient is estimated from conventional formulas, (2) adjustments are made to that estimate according to patient specific details, and (3) further adjustments are made according to continuous glucose data obtained during the evaluation period in order to bring the patient's glucose into a euglycemic range. By the end of the evaluation period, by which time substantially normal glucose values have been achieved, various quantitative relationships from accumulated data are calculated and then applied to determining insulin dosages for an ensuing extended period of therapy. Through the implementation of appropriate insulin dosing, diabetic patients can achieve glucose profiles through the day that are substantially similar to those of non-diabetic people, and without a clinically significant rate of occurrence of hypoglycemic episodes.

The individualized constants that account for the balance among carbohydrate consumed, glucose level, and insulin dosages that are determined during the evaluation period characterize the patient at that point in time. In the absence of unusual events or disease processes, the values obtained during the evaluation period provide clinically appropriate insulin dosages that remain constant typically over a period of at least several months, for example, at least for a period of about 3 to about 6 months. If unexpected deviations from euglycemia emerge during a period of therapy, the evaluation regimen embodied in the method may be repeated, and a new dosage schedule applied over a subsequent therapeutic period.

Embodiments of the invention relate to assessment and education of the patient, and may include determination as to whether the patient is an appropriate candidate for the CGIA method. Patient appropriateness is determined by such criteria as the patient's physical and sensory abilities, such as vision and hearing, psychological state, stable insulin sensitivity, and capacity for compliance with instructions. Patients may be further trained with regard to carbohydrate counting, i.e., gaining an ability to accurately estimate the carbohydrate content of a meal. Patients may be trained with regard to insulin treatment with either multiple daily injections (MDI), typically four or more self-administered injections/day, or an insulin pump and the use of self-monitored glucose testing with a properly function meter, as is appropriate to the type of treatment the patient is receiving. Patients are educated in a conceptual sense with regard to the underlying theory of basal insulin infusion rates and bolus dosing, the need for ideal glucose control, and an operating knowledge of the insulin pump.

Embodiments of the invention thus include an initial evaluation period in the context of a structured outpatient program, during which time patient-individualized insulin dosing and dosing factors based on a series of daily continuous glucose monitor (CGM) downloads and consequent insulin dose adjustments are established. Embodiments of the invention also include a structured and tracked or diary-recorded program of diet and activity during the treatment period.

As mentioned above, embodiments of the invention include implementation of a structured and tracked program of diet and activity during the treatment period. Thus, for example, patients may maintain a diary in which notes are kept of activity, eating, insulin dosing, and the occurrence of diabetes-related symptoms. The practice of having a structured eating and activity plan, and maintaining a diary to record relevant information may begin during the evaluation period before formal treatment with methods described herein is initiated. A function of the diary is to provide a context with regard, for example, regarding diet and activity, within which to understand and interpret the dynamics of insulin dosing and glucose values.

Embodiments of the invention further include daily analysis of the CGM tracing with adjustments computed by mathematic formulas in a sequence of establishing of a basal insulin infusion rate first, then the determining the proper carbohydrate to insulin ratio, and finally, determining the correction factor. Daily analysis of the CGM tracing can be understood as an analysis of the patient glycemic condition, the levels of glucose as reflected in data that are collected continuously over the course of the evaluation period. Embodiments may include adjustment of the current or a conventional estimate of insulin dosing, if needed, according to individual medical aspects of the patient. During the evaluation period, when continuous glucose data are being reviewed daily by a healthcare professional, and adjustments to insulin doses being made, glucose data that follow any change in insulin dosage are specifically followed for a period of 48 hours in order to check that a patient-specific goal of glucose control has been met. Implementation of the method is not complete until such a 48 hour period of appropriate glycemic control has been achieved. As mentioned elsewhere, this goal of 48 hours of glycemic control is typically achieved in about seven days.

One particularly problematic aspect of determining a clinically appropriate basal level of insulin dosing by conventional methods is the avoidance of hypoglycemic episodes that can occur as a consequence of missing a meal. Such an episode is likely to occur when the basal insulin dosage or rate of infusion is too high. Accordingly, an aspect of the invention includes the deliberate missing of a meal, typically, separately missing one each of breakfast, lunch, and dinner, during the course of the evaluation period. By such deliberate missing, periods of hypoglycemic vulnerability are created, and insulin dosages are determined by the method during the evaluation period with particular attention to avoiding hypoglycemic episodes.

In some embodiments, the collection and analysis of data from the CGIA evaluation period are performed in the office a diabetes specialist; in other embodiments, the patient interacts with a physician or health profession remotely via the internet. In such embodiments where a health care professional expert in continuous glucose monitoring is remotely engaged, a patient gathers the information, transfers it electronically, via the internet, for example, and the data are then interpreted by the health care professional, who and makes changes in insulin treatment, and transmits such changes to the patient, or directly to the control systems that operate the patient's insulin pump.

Aspects of the invention were applied in a prospective study (King and Armstrong, Journal of Diabetes Science and Technology 1, 36-46, 2007) that included thirty type 1 diabetic patients without a functioning pancreas, who received an outpatient adjustment of their insulin dosing by their insulin pump that guided by data from CGM with near daily uploads and insulin adjustments. As exemplified by this study, this embodiment of the procedure takes about 30 minutes per day, about seven office visits with minimal alterations in eating, activity and life activity. In the exemplary study, a composite basal glucose analysis showed the mean glucose level to be 115 mg/dl, well within the euglycemic standard (glucose<130 mg/dl) of the American Diabetes Association After adjustment of the bolus insulin dosing formulas, the mean post-meal glucose values returned to within 10% of target glucose. At a follow up examination (occurring at a mean of 12 weeks after implementation of the adjusted insulin dosage), the average AlC level had decreased 0.5% (both statistically and clinically significant) in those subjects with a AlC at baseline of >7.0%.

Embodiments of the invention make use of the application of quantifiable relationships among the total daily dose of insulin, the total basal dose of insulin, carbohydrate consumed, the insulin-to-carbohydrate ratio, and the correction factor or insulin sensitivity ratio, as determined during the patient's evaluation period. These terms are used according to the conventions that follow below.

The total daily dose of insulin (TDD, units/d) is the total amount of insulin administered per day. TDD equals the total basal dose (TBD, see below) plus the total bolus doses delivered per day. Changes in the meal carbohydrate content change the required total bolus dose, and consequently the TDD.

The total basal dose of insulin (TBD, units/d) is the total number of units of insulin administered per day to maintain normal glucose levels during fasting. Normal glucose control includes prevention of hyperglycemia due to the early morning increase in insulin resistance (the so-called “dawn phenomenon”) as well as avoidance of hypoglycemia when a meal is omitted.

The insulin to carbohydrate ratio (ICR, grams of meal carbohydrate/unit of insulin) is a patient-specific constant which, when divided into the grams of carbohydrate to be eaten, yields the amount (or units) of rapid acting insulin (RAI) that will return the patient's glucose value to the pre-meal glucose level within four hours post-meal.

The correction factor (CF, mg/dl/unit) is also known as the insulin sensitivity ratio or insulin sensitivity factor. This is a patient-specific constant which, when divided into the difference between an elevated and the target glucose (mg/dl), yields the number of units of rapid acting insulin (RAI) needed to return the glucose to target level (usually 100 mg/dl) within four hours.

The mathematical relationship between the total basal dose of insulin (TBD) and the total daily dose of insulin (TDD) was determined in an exemplary prospective study of 30 patients that followed a protocol of selecting patients by positive criteria and excluding by negative criteria, as described in detail below. From the data shown in FIG. 2, the slope of the line describing the TBD (on the Y axis) as a function of TDD (on the X axis) was determined to be: TBD=0.387×TDD.

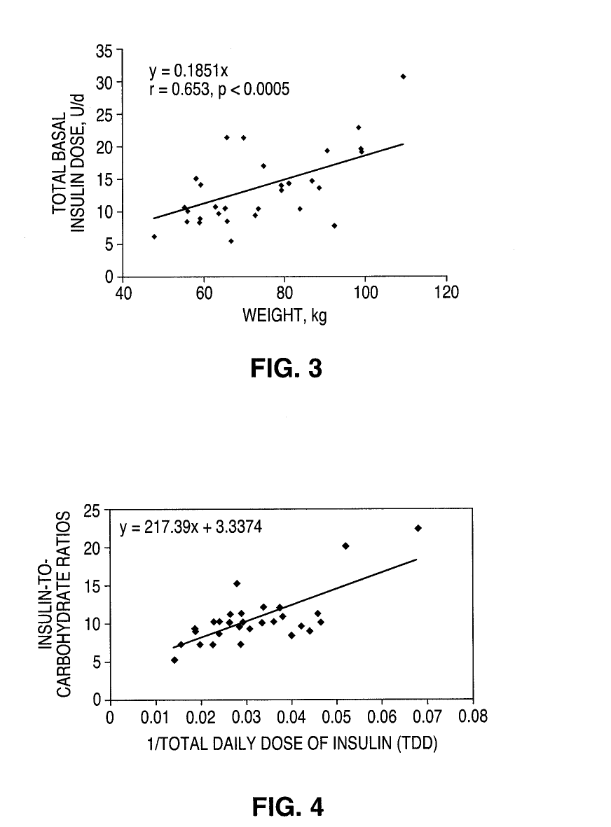

The relationship between the total basal dose of insulin (TBD) and the total daily dose of insulin (TDD, U/day) and the patient's body weight (kg) was also observed in the study described above. From the data shown in FIG. 3, the slop of the line describing TBD (on the Y-axis) as a function of weight was determined to be: TBD 0.185×Kg.

The relationship between the insulin-to-carbohydrate ratio (ICR) and the total daily dose (TDD) was observed in the study described above. From the data shown in FIG. 4, the equation of the line describing ICR (Y axis) as a function of the reciprocal of TDD (X axis) was determined to be: ICR=217×1/TDD+3.3.

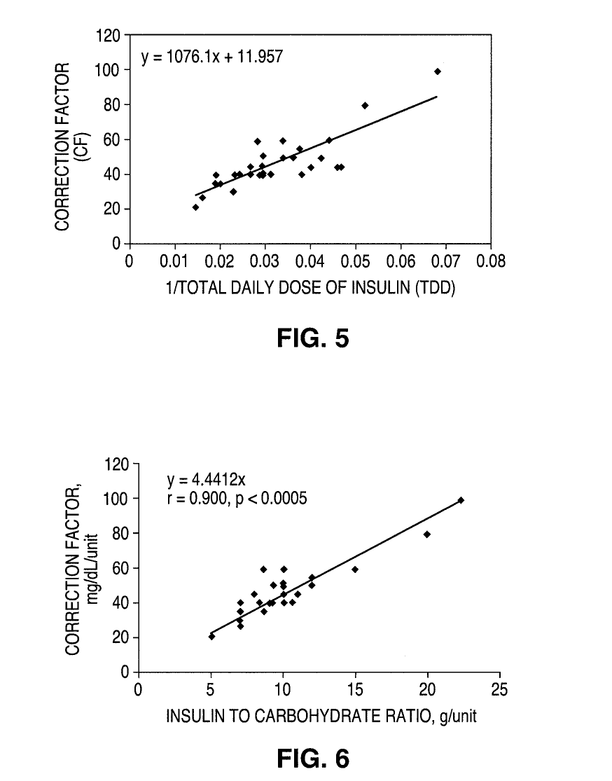

The relationship between the correction factor (CF) and the total daily dose (TDD) was observed in the study as described above. From the data shown in FIG. 5, the equation of the line describing the CF (Y axis) as a function of the reciprocal of the TDD (X axis) was determined to be: CF=1076×1/TDD+12.

The relationship between the correction factor (CF) and the insulin-to-carbohydrate ratio (ICR) was observed the study as described above. From the data shown in FIG. 6, the equation of the line describing the CF (Y axis) as a function of the ICR (X axis) was determined to be: CF=4.44×ICR.

These preceding equations are non-limiting examples of a set of observations that allowed a reasoned estimation of the fundamental quantitative relationships among TBD, TDD, carbohydrate consumption, ICR, and CF for the patients in the referenced prospective study. The various specific formulas or values for constants that are used in the description of embodiments of the invention, but they are intended only as non-limiting examples or estimates of the underlying fundamental relationships. Accordingly, other estimations or approximations of these values may be made, and may be appropriately used in the practice of this invention without departing from the scope of the invention. Further exemplary or illustrative, and not limiting, are specific values of glucose levels, such as clinical target values or any clinical value generally characterized as hypoglycemic, euglycemic, or hyperglycemic.

As shown in FIG. 1, embodiments of the method of appropriately adjusting insulin-dosing include a general flow sequence of (1) making or accepting an initial dose estimation, (2) making a pre-dose adjustment, and (3) making a post-dose adjustment, as detailed below.

Dose estimation: From the patient's weight or total daily dose (TDD), the TBD, the ICR, and the CF are estimated from formulas as follows:

TBD=0.185*weight (in kg), or alternatively, TBD=0.384*TDD (in units/d) a.

ICR=(217/TDD)+3, or alternatively, ICR=(59/TBD)+5 b.

CF=4.44*ICR, or alternatively, CF=(1076/TDD)+12, or =(276/TBD)+22 c.

Pre-Dose Adjustment: Before beginning insulin treatment, the dose or dosing factors may be adjusted according to the clinical estimation of the patient's insulin sensitivity. For example, the dose may be increased in for an obese patient or one being treated with prednisone, or decreased in patients of advanced age or with renal failure.

Post-Dose Adjustment: After beginning insulin treatment, the dose or dosing factors may be adjusted based on assessment of symptoms (hyperglycemia or hypoglycemia), or from self monitored glucose (SMBG) data or continuous glucose monitoring (CGM) data. The method described herein, termed “continuous glucose monitoring-directed insulin adjustment” (CGIA) utilizes CGM for glucose sensing data, and mathematical formulas for adjustment of insulin dosing.

Embodiments of the invention include the exercise of both positive and negative criteria for ensuring that the therapeutic method is applied only to suitable patients, suitable in terms of ability to participate in the therapy, and suitable in terms of ability to benefit from the therapy. Some embodiments of the invention include methods of selecting patients that are appropriate for the therapeutic method as represented by the exemplary positive criteria that relate generally to patient capacity to manage the basic aspects of diabetes treatment, effective management relying on understanding of the medical principles and physical and mental capability to operate the therapeutic method. Examples of such criteria follow below.
1. Patient is familiar with basic diabetes management (as shown, for example, by successful completion of a basic diabetes education course).
2. Patient is capable of carbohydrate counting, as demonstrable, for example, by successful completion of a basic carbohydrate counting course.
3. Patient is knowledgeable about basal and bolus insulin dosing. The preferred level of knowledge typically includes a basic conceptual understanding of insulin and the effects it has on glucose levels, and for example, the use of dosing formulas, and “insulin on board” rule.
4. Patient is capable of self-monitoring glucose four or more times per day. For example, it is preferable that the patient be able to use the control solution and the meter time/date set function correctly.

Some embodiments of the invention may include methods of excluding patients that may be physiologically, mentally, or psychologically inappropriate for the therapeutic method, as represented by the exemplary excluding criteria that follow below. Patient exclusion criteria may be broadly characterized as relating to (1) mental factors, such as state of knowledge, attitude, or behavior, and (2) medical factors, such as physical or sensory disability, insulin instability, or any complicating, unpredictable, or emerging condition that may affect the relationships among carbohydrate consumption, glucose, or insulin sensitivity. Exclusionary criteria may be framed in terms that describe an appropriate patient in that it may be said that the patient does not have such mental or medical characteristics.

Examples of mental characteristics that would typically preclude a patient from participating in a continuous glucose data-based insulin adjustment evaluation may include the following:
1. Patient has demonstrated noncompliance or noncompliant tendencies, as could be observed, for example, with regard to clinic attendance and instructions.
2. Patient does not have sufficient knowledge or practical mastery of use of the infusion pump (if using CSII, continuous subcutaneous insulin infusion). The level of knowledge and practice are evaluated with particular attention to problem solving, such as, for example, ability to bring up basal rate screen, setting bolus wave forms, and how to respond to various alarms.

Examples of medical characteristics that would typically preclude a patient from participating in a continuous glucose data-based insulin adjustment evaluation include the following:
1. Patient has a physical or sensory impairment, such as being hearing impaired or visually impaired, or impaired in any way that disallows operation of devices or materials required for the therapy. Exclusion of hearing and visually impaired patients follows, for example, from the fact that auditory and visual ability are required to operate a continuous glucose monitoring system, such as, merely by way of example, the CGMS® Gold system of Medtronic Minimed (Northridge Calif.). In the eventuality that a continuous glucose monitoring system becomes available that allows patients with such a physical or sensory impairment to operate the system, then the impairment would no longer represent an exclusionary factor.
2. Patient shows clinical indications of unstable insulin sensitivity or the patient is considered to be at risk for such instability, as would be expected of patients, for example, who are starting prednisone treatment, contemplating a weight loss program, under severe stress, planning major surgery, or hosting a moderate or severe infection.
3. Patient has demonstrated an apparently unstable eating or activity patterns, or a history thereof.
4. Patient has gastroparesis or history thereof. Gastroparesis, a condition of erratic gastric emptying, is associated with unpredictable post-prandial glucose excursions.

Some embodiments of the invention further include generally preparing patients for the therapeutic method according to various guidelines that apply to the pre-therapeutic evaluation period, and continued adherence to such guidelines during therapy. The development of a structured life pattern with regard, for example, to sleep and wakefulness, physical activity, and eating, is beneficial with regard to achieving a successful evaluation period. Patients are typically placed on a structured life pattern (outlined below) during the insulin adjustment period in order to establish the proper basal insulin rate and bolus ratios. Once the period of testing is completed, and the rates and ratios are established, the typical patient returns to his or her general pattern of activity. For best results during insulin therapy, of course, continued diligence with regard to carbohydrate counting and other aspects of a structured pattern of daily activity is beneficial.

Practice of the method and ensuing therapy is benefited by good patient compliance with guidelines as described herein, however the standard for compliance should not become perfection, lest the goal of perfection become the enemy of the good. Further guidelines thus relate to the patient developing or adopting an appropriately balanced attitude and practice with regard to rigor, realism, estimation, and approximation that is helpful for achieving successful compliance with the therapy. Patients are educated to understand that there is an acceptable degree of approximation in carbohydrate counting, calculations, and glucose control. Sources of error and variability include, for example, the fact that typical glucose determinations include an error of ±10% of true value, carbohydrate estimation has an error, the amount and rate of insulin absorption from a subcutaneous site is physiological variable, and the rate of absorption of glucose from the meal is a physiological variable. These considerations notwithstanding, the more these variables are controlled or minimized during the (CGIA) evaluation period, the more dependable and appropriate will be the dosing calculations, and the more efficacious will be the therapeutic method.

Some embodiments of the invention include guidelines for patients during the evaluation period with regard to eating habits, such guidelines may be appropriately extended into the therapeutic period, but for the purposes of describing the method, the method substantially relates to aspects of the evaluation period that precede the subsequent therapeutic period. General eating guidelines include the stipulations that there should be meal consistency (for breakfast, lunch, and dinner, respectively, with regard to the food content, and the time of the meal during day), that all food must be weighed accurately, preferably by an electronic weight scale, and that a hypoglycemic episode be treated with glucose tablets (4 gm tablets, for example).

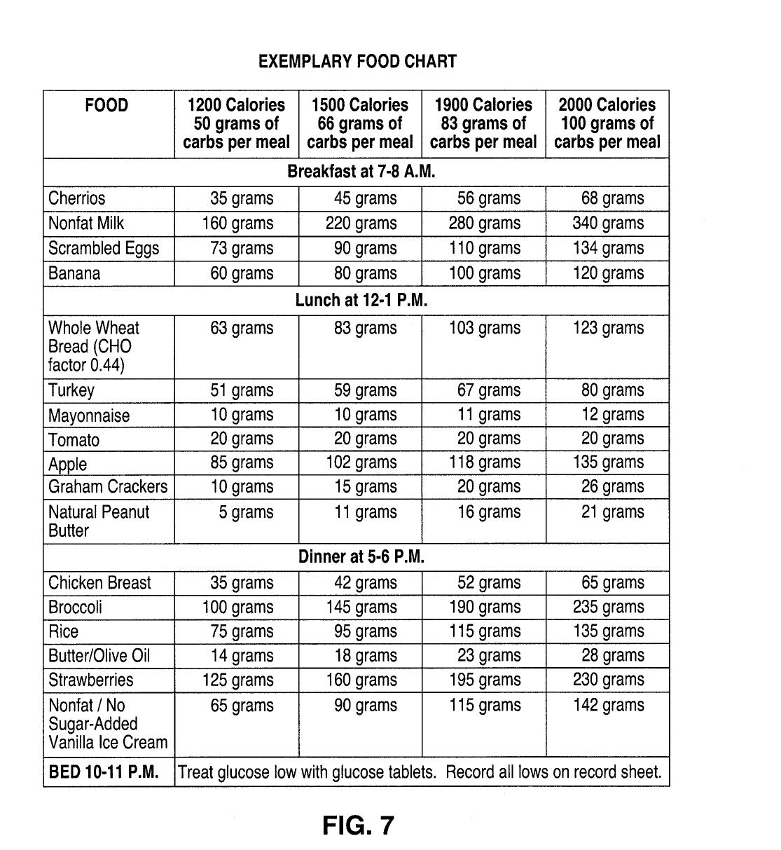

Further, some embodiments of the evaluation period of the method include the patient maintaining substantial caloric consistency, while accommodating a degree of variation in types of food eaten. This isocaloric goal is aided by the use of a “food selection chart” such as the example provided by FIG. 7, which provides a guide for a diet that is 50% carbohydrate, 30% fat and 20% protein diet. More specifically, an isocaloric aspect of an embodiment of the method entails the patient eating the same amount at each respective meal, such as the same amount at each breakfast from day-to-day, at each lunch day-to-day, and at each dinner day-to-day. With the same amount being eaten at each respective meal, the same total daily amount is eaten from day-to-day. An exception to the isocaloric consumption from day to day is represented by the days when a meal is omitted (as provided by embodiments of the invention) for the purpose of observing the glucose response, more particularly to ascertain whether the patient becomes hypoglycemic. When a meal is omitted, the total caloric consumption for that day is less than a day in which every meal is included.

Eating guidelines also may include eating an appropriate number of calories during the day, such that calories consumed in the form of food does not exceed the number of calories expended by the patient. An equation, such as for example, the Harris Benedict equation (Harris and. Benedict “A biometric study of basal metabolism in man”, Washington D.C. Carnegie Institute of Washington, 1919) may be used to calculate the number of calories expended per day, a value known as the basal metabolic need (BMN). Other equations that serve substantially the same purpose as the Harris Benedict equation may be used in the practice of embodiments of this invention.

Variations of a basic metabolic need equation (such as the Harris Benedict equation) may be appropriately applied to broad classes of patients that are known to have metabolic differences as a class, such as do women and to men, by including activity multipliers that may be factored into the equation. For women, for example, the BMN may calculated from the exemplary formula:

BMN=655+(9.6×weight in kg)+(1.8×height in cm)−(4.7×age in years)

For men, for example, the BMN may be calculated from the exemplary formula:

BMN=66+(13.7×weight in kg)+5×height in cm)−6.8×age in yrs)

Various activity-based multipliers also may be applied to the BMN, as illustrated by the following examples:

a. If sedentary (little or no exercise, desk job): multiply the BMN by 1.2.

b. If lightly active (light exercise/sports 1-3 days/wk): multiply by 1.375.

c. If moderately active (moderate exercise/sports 3-5 days/wk): multiply by 1.55.

Some embodiments of the invention include guidelines for patients with regard to their level of activity. These guidelines apply particularly to patient activity during the evaluation period, although they may further be applied to activity during the extended therapeutic period following the evaluation period. Wearing the CGMS monitor typically restricts physical activity to a level that ranges between less-than-moderate and moderate. If a patient exercises, he or she should exercise with consistency, as for example, by exercising at the same time during the day, and with the same intensity and duration each day.

Changes in sleep cycle can change the required basal insulin infusion rate; thus, some embodiments of the invention include a guideline that there should be minimal changes in sleep pattern of patients being treated by the method. These guidelines apply particularly to patient activity during the evaluation period, although they may further be applied to activity during the extended therapeutic period following the evaluation period.

Embodiments of the invention include a provision for resetting the insulin basal rate and bolus ratios before beginning the evaluation period of the method, as shown in FIG. 1. If either the pre-study basal rate or bolus ratios markedly deviate from those that would be calculated from conventional formulas, the insulin dosing should be adjusted prior to beginning the evaluation period of the method.

Some embodiments of the invention address the class of patients entering the evaluation period that are already on an insulin pump, and making adjustments to their dosage schedule prior to the evaluation period. Thus, in those patients already on insulin pump treatment and with regard to the basal insulin infusion rate, if the total basal dose (TBD) is greater than 50% of the total daily dose (TDD), then the TBD may be adjusted to a level that is 40% of TDD. With regard to the insulin to carbohydrate ratio (ICR), if it differs by more than 50% from the formula (ICR=(59/TBD)+5) once the TBD has been corrected, as above, then the ICR formula may be adjusted such that ICR=59/TBD+5. In this case, the CF may also be changed by using the formula CF=4.44*ICR.

In those patients not currently on an insulin pump but who are converting to an insulin pump from multiple daily insulin injections (MDI) the dosing may set initially set by the exemplary procedure that follows.
a. Calculate the TBD with reference to either to the TDD (TBD=0.185*Wt (kg) or body weight (TBD=0.384*TDD) and go forward with the lower of the two values.
b. Divide the calculated TBD by 24 to yield the average basal insulin rate (units/hr).
c. From the time point starting at one hour before usual onset of sleep and extending through to a time point one hour before usual awakening, deliver insulin at 1.4*average basal rate.
d. At all other time, deliver insulin at 0.8*average basal rate.
e. Set ICR at 59/TBD+5.
f. Set CF at 4.44*ICR.

Some embodiments of the invention include performing insulin dose adjustments under various conditions, based on continuous glucose monitoring (CGM) data during the evaluation period (as per FIG. 1). In the event that the CGM finding is that the basal glucose level is out of range, adjustment of the insulin is made according to formula 1.

Δ basal rate¹=(ΔBG²/CF³)/5−(g_{GT}⁴/ICR⁵)/5

In the event that the CGM finding is that the fourth hour postprandial glucose level is out of range, adjustment of the insulin is made according to formula 2.

ICR_{new}=[gₘₑₐₗ⁶+(ΔBG_{ac}⁷/4.44)]/[units given⁸+(ΔBGₚₒₛₜₘₑₐₗ⁹/CF³)]

CF_{new}=4.44*ICR_{new}

A key to the terms of these preceding formulas is as follows.
1. Δ basal rate=the amount that the basal rate is changed (+ or −); the units of the term are units of rapid acting insulin (RAI) per hour). The change in the basal rate change typically begins about one hour before the beginning of the glucose being “out of range” to about one hour before the peak of the glucose change.
2. ΔBG=the difference between the maximum glucose that is “out of range” minus the target glucose level (usually 100 mg/dl) during the period being analyzed.
3. CF=Correction factor; the units of the term are mg/dl/unit of rapid acting insulin (RAI). The CF is derived as follows: CF=4.44*ICR.
4. g_{GT}=grams of glucose consumed from glucose tablets for treating hypoglycemia.
5. ICR=Insulin-to-carbohydrate ratio (grams of meal carbohydrate/unit of RAI). The ICR is either known or derived from TBD, as follows: ICR=59/TBD+5
6. gₘₑₐₗ=grams of meal carbohydrates eaten.
7. ΔBG_{ac}=the difference between the glucose level prior to the pre-meal bolus and the target level (typically 100 mg/dl).
8. Units given=the total number of units of RAI bolus given before the meal.
9. ΔBGₚₒₛₜₘₑₐₗ=the difference between the glucose level at the fourth post bolus hour and the target level (typically 100 mg/dl).

In analyzing patient data according to embodiments of the invention, a series of evaluation steps may be taken, as follows:
1. Establishing the basal rate: Begin by dividing the basal dose into various appropriate time periods encompassing the day. An exemplary series of daily time segments is represented by the four periods substantially defined by meal and sleep schedule: (1) breakfast to lunch, (2) lunch to dinner, (3) dinner to bed, and (4) bed to breakfast.
2. Omit meals in the daily sequence of supper, lunch and then breakfast and adjust per above the corresponding basal rates during these periods to target glucose levels. Start with the basal rate period four hours after the lunch meal to the breakfast meal of the next day. One of the purposes of omitting meals is to test against the possibility that the insulin dose is too high, such that in the absence of a recent meal, the glucose level drops to a clinically unacceptable low level. In the event of such a hypoglycemic excursion, a healthcare professional (as provided by embodiments of the invention) makes a compensatory adjustment of the insulin dosage.
4. Evaluate the carbohydrate to insulin ratio (ICR) at any meal; this may be done contemporaneously with the basal testing period.
5. Once ICR is established, determine the correction factor (CF) by the formula, CF=4.44*CIR.

A formula for estimating the ICR is as follows: ICR=217/TDD+3. Based on observations of the inventor, there appears to be no significant difference between ICR over the course of the day, therefore the same ICR may be used for all meals. An example of the application of the ICR calculation is as follows: A patient is to consume 100 grams of carbohydrate with a meal. Assume the ICR is 10 grams of carbohydrate per unit of rapid acting insulin (e.g., lispro, aspart or glulisine). Therefore the insulin units to be injected=100/10=10 units.

A formula to calculate the correction factor (CF) is as follows: CF=4.44×ICR. An example of the application of the CF calculation is as follows: If the current glucose prior to a meal is 200 mg/dl, the target is 100 mg/dl and the CF is 50 mg/dl/unit of RAI, then, the units to be injected=(200−100)/50=2 units. As with ICR, the CF appears to be the same for all meal times.

There is a mathematical relationship between ICR and CF, as follows: CF=4.44*IC. For example if the ICR is 8 g/unit then the CF would be: 4.44*8=˜35 mg/dl/unit. The target glucose is typically placed at 100 mg/dl. This glucose target level may be adjusted upward in those patients who are considered to be relatively intolerant of hypoglycemia.

Some embodiments of the invention include educational and training materials related to the method; in some embodiments, the materials may be directed toward the patient; in other embodiments, the materials may be directed toward the health care professionals who are caring for the patients. Educational and training materials may be presented in any one or more forms, such as written materials (booklets, pamphlets, wall charts), or video or audio recordings. Some embodiments of the invention may include data recording and calculation tools. These data-related tools may include paper forms or charts that can be filled in by the patient or health care professional. Some embodiments may include electronic files, spreadsheets, for example, into which data may be entered. Such electronic files may have formulas built-in for operational convenience, such as ease and accuracy in handling data, making calculations, and charting relationships within the data.

Some embodiments of the invention include an integration of aspects of data collection and calculations based on the data into the software of an insulin infusion pump in order to control the insulin dosages. Embodiments of the invention may be practiced without limitation regarding the use commercially available and FDA approved insulin pumps or continuous glucose monitors. In some embodiments where a patient is making use of both an insulin pump and a continuous glucose monitor, particularly when the insulin pump and the glucose monitor are integrated into a closed loop system, data collection and calculation software that facilitates the practice of the invention may be distributed into appropriate sites within the combined system. In addition to glucose values as data input into the calculations that adjust the insulin dosages, the calculations may also include data entered by the patient regarding the carbohydrate count of food that has been consumed.

### EXAMPLES

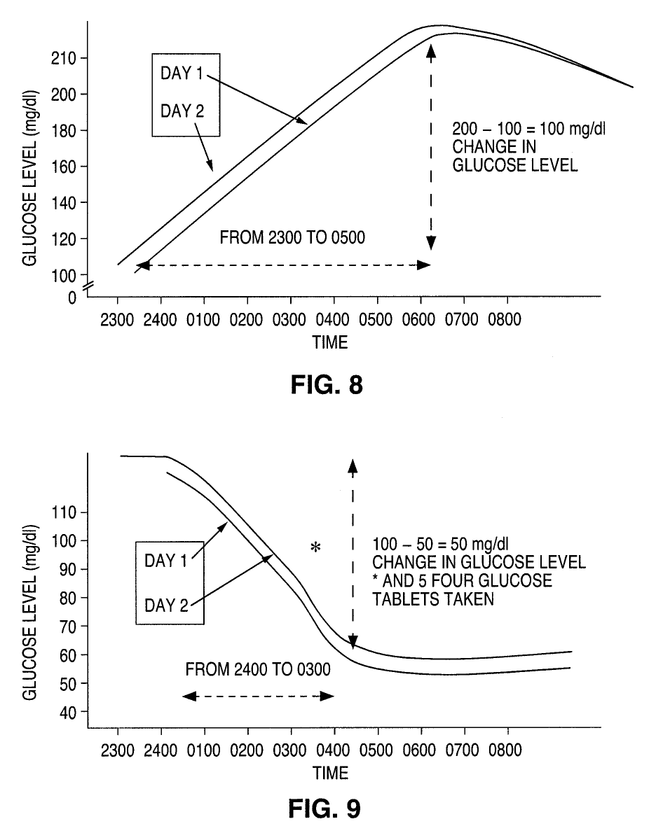

Examples of the application of the inventive method are now provided in FIGS. 8-11. FIGS. 8 and 9 provide examples of data and analysis per the inventive method from hypothetical patients in which, respectively, basal glucose is too high (FIG. 8) or too low (FIG. 9).

With reference to FIG. 8, it appears that the patient's basal level of glucose is too high. The analysis begins with the question: “is there a glucose pattern?” A review of the data would note that the pattern is repeated for two days. The elevated glucose depicted in FIG. 8 could be due an inadequate meal insulin bolus or from inadequate insulin basal rate. Omitting this meal will allow the isolated evaluation of an inadequate insulin basal rate. The patient's diary notes should be checked for any other event that may have taken place during this time.

In FIG. 8, the pattern shows glucose elevated to a peak level of 200 mg/dL during the time period 2300 to 0500 hours. The basal insulin infusion rate can then be corrected according to the formula, assuming for example the TBD is 10 units/day, an ICR of 10 g/unit and a CF of 45 mg/dL/unit, and no glucose tablets for hypoglycemia were taken, as follows:

Δ basal rate=(ΔBG/CF)/5−(g_{GT}/ICR)/5

Δ basal rate=((200−100)/(45))/5−((0/ICR)/5)

Δ basal rate=(100/˜50)/5−(0)/5

Δ basal rate=0.4−0=0.4 units/hour added from 22:00 hours to 04:00 hours (hour before the start of the increase to one hour before the peak of the increase).

With reference to FIG. 9, it appears that the patients' basal level of glucose is too low. The analysis begins with the question: “is there a glucose pattern?” The low glucose depicted in this FIG. 9 could be due an excessive meal insulin bolus or from inadequate insulin basal rate. Omitting this meal (in this example, the dinner) and its bolus will allow the isolated evaluation of the insulin basal rate. Check the patient's diary for any other event that may have taken place during this time.

In summary, the pattern shows the glucose level of 50 mg/dL is lower than the target range, and that this level occurs from 24:00 to 03:00 hours. In addition, five 4-gm glucose tables were ingested. The basal insulin infusion rate can then be corrected according to the formula, assuming a TBD of 10 units/day, ICR of 10 g/unit and CF of 45 mg/dL, unit, as follows:

Δ basal rate=(ΔBG/CF)/5−(g_{GT}/ICR)/5

Δ basal rate=((50−(100/45))/5−(5 tabs*4 grams each)/10)/5

Δ basal rate=(−50/˜50)/5−(20/10)/5=−0.2−(2.0)/5=−0.2−0.4=−0.60 units/hr
Δ basal rate 0.60 units/hour subtracted from the current basal rate between 23:00 H to 02:00 H (one hour less than the start of the fall in glucose and one hour less than the hour of the lowest glucose).

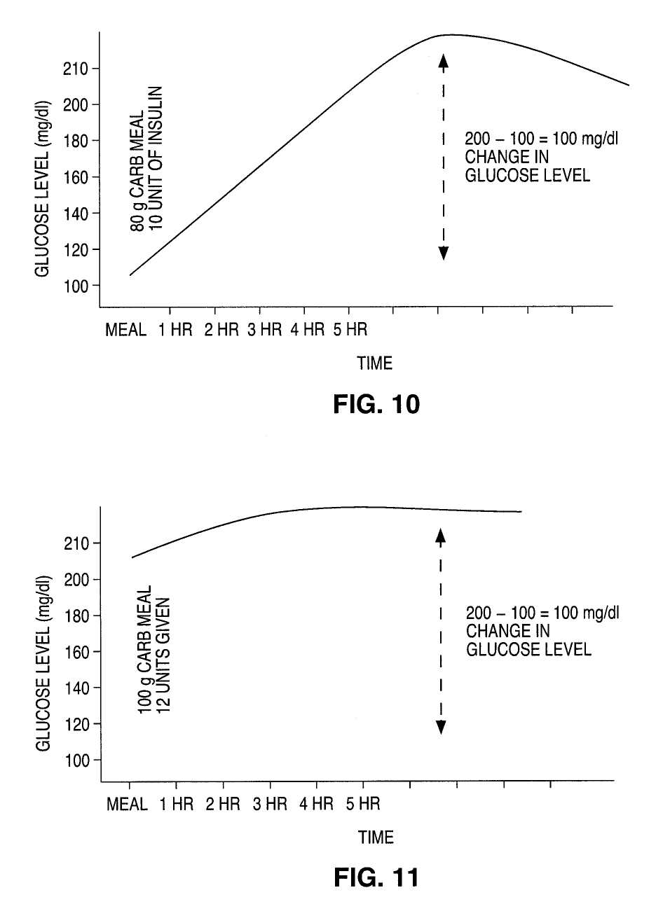

With reference to FIG. 10, analysis of the data shows that although the premeal glucose was at a target level, the post meal glucose was elevated. Assuming that the TBD is 10 units/day, the TDD is 20 units/d, the meal carbohydrate content is 80 g, and that 10 units of bolus insulin is given, the ICR may be calculated as follows:

ICR_{new}=[gₘₑₐₗ+(ΔBG_{ac}/4.44)]/[units given+(ΔBGₚₒₛₜₘₑₐₗ⁹/CF³)]

ICR_{new}=[80+((100−100)/4.44))]/[10+((200−100/(59/TBD+5)))]

ICR_{new}=[80+(0/4.44)]/[10+(100/55)]

ICR_{new}=80/[10+≈2]=80/≈12=6.7 g/unit

CF_{new}=4.44*ICR_{new}=4.44*6.7=29.7 or 30 mg/dl/unit

With reference to FIG. 11, both pre-meal and post-meal glucose are elevated. Assuming that the starting TBD, TDD and meal carbohydrate content but an elevated pre meal glucose of 180 mg/dl, the insulin to carbohydrate ration may be calculated as follows:

ICR_{new}=[gₘₑₐₗ+(ΔBG_{ac}/4.44)]/[units given+(ΔBGₚₒₛₜₘₑₐₗ⁹/CF³)]

ICR_{new}=[80+(180−100/4.44)]/[12+((200−100)/((1076/TDD)+12))]

ICR_{new}=[80+˜18]/[12+(100/˜55)]

ICR_{new}=98/[12+2]=98/14=7 g/unit

CF_{new}=4.44*ICR_{new}=4.44*7=31.1 or 31 mg/dl/unit

### Terms and Conventions

Unless defined otherwise, all technical terms used herein have the same meanings as commonly understood by one of ordinary skill in the art of treating diabetes. Specific methods, devices, and materials are described in this application, but any methods and materials similar or equivalent to those described herein can be used in the practice of the present invention. While embodiments of the invention have been described in some detail and by way of exemplary illustrations, such illustration is for purposes of clarity of understanding only, and is not intended to be limiting. Various terms have been used in the description to convey an understanding of the invention; it will be understood that the meaning of these various terms extends to common linguistic or grammatical variations or forms thereof. It will also be understood that when terminology referring to devices, equipment, or drugs has used trade names, brand names, or common names, that these names are provided as contemporary examples, and the invention is not limited by such literal scope. Terminology that is introduced at a later date that may be reasonably understood as a derivative of a contemporary term or designating of a subset of objects embraced by a contemporary term will be understood as having been described by the now contemporary terminology. Further, while some theoretical considerations have been advanced in furtherance of providing an understanding, for example, of the quantitative interrelationships among carbohydrate consumption, glucose levels, and insulin levels, the claims to the invention are not bound by such theory. Moreover, any one or more features of any embodiment of the invention can be combined with any one or more other features of any other embodiment of the invention, without departing from the scope of the invention. Still further, it should be understood that the invention is not limited to the embodiments that have been set forth for purposes of exemplification, but is to be defined only by a fair reading of claims that are appended to the patent application, including the full range of equivalency to which each element thereof is entitled.

What is claimed is:

1. A method of treating a diabetic patient with an insulin dosage schedule clinically appropriate for that individual patient comprising:
testing the patient to determine the patient's carbohydrate-to-insulin ratio (CIR) and the correction factor (CF) during an evaluation period under conditions of structured daily activity;
applying the CIR and CF to determine a clinically appropriate basal insulin dosage and insulin bolus dosage; and
applying the insulin dosages to the patient for a period of insulin therapy.

2. The method of claim 1 wherein testing to determine the CIR and CF includes making a first estimate of an appropriate insulin dosage schedule based on available information, using that estimate at the outset of the evaluation period, and making adjustments to the first estimated dosage schedule based on continuous glucose data obtained during the evaluation period.

3. The method of claim 2 wherein estimating an appropriate insulin dosage includes making a first estimate of the total basal dosage based on a formula based on any of patient weight or on the existing total daily dosage schedule for the patient.

4. The method of claim 3 wherein making a first estimate further includes adjusting the estimated basal dosage to account for any other clinical factors that may affect insulin sensitivity.

5. The method of claim 2 wherein testing to determine CIR and CIF during an evaluation period includes making adjustments to the first estimated dosage schedule based on data obtained during the evaluation period.

6. The method of claim 5 wherein the data include any one or more of observed symptoms of hyperglycemia, observed symptoms of hypoglycemia, or glucose data obtained from a continuous glucose monitor.

7. The method of claim 1 wherein structured daily activity includes a controlled pattern of eating, a controlled pattern of physical activity, and a controlled pattern of sleep.

8. The method of claim 7 wherein controlled daily activity comprises moderation and consistency with regard to the daily activity.

9. The method of claim 8 wherein consistency with regard to eating includes eating a consistent number of calories from day to day.

10. The method of claim 8 wherein moderation with regard to eating includes consuming a number of calories daily that is about equal to the number of calories expended daily.

11. The method of claim 8 wherein consistency with regard to eating includes eating a same number of calories at each breakfast from day-to-day, at each lunch day-to-day, and at each dinner day-to-day.

12. The method of claim 8 wherein consistency with regard to eating includes eating meals about the same time every day.

13. The method of claim 8 wherein consistency with regard to physical activity includes exercising at about the same time and at about the same amount every day.

14. The method of claim 1, further comprising accepting the individual as an appropriate patient for the therapy by applying screening criteria.

15. The method of claim 14 wherein the screening criteria include positive selection criteria related to managing the basic aspects of the treating diabetes.

16. The method of claim 14 wherein the screening criteria include exclusionary selection criteria.

17. The method of claim 1, further comprising educating the patient regarding medical components of the therapy.

18. The method of claim 1 wherein the screening criteria include positive criteria for patient selection, such positive criteria including any one or more of familiarity with basic diabetes management, capability to count carbohydrates, knowledgeable about basal and bolus insulin dosing, or capability to self-monitor glucose.

19. The method of claim 1 wherein the screening criteria include exclusionary criteria for patient selection, such exclusionary criteria including any one or more of demonstrated noncompliance, an impairment that comprises an ability to operate the method, an insufficient knowledge of how to use of an infusion pump (if using a pump), a clinical indication of unstable insulin sensitivity, an unstable eating or activity patterns, or a diabetes-relevant complicating condition of the digestive tract.

20. The method of claim 1 further comprising complying with guidelines regarding the structured daily activity, the patient exercising the compliance.
